(19) United States
(12) Patent Application Publication
(10) Pub. No.: US 2026/0008809 A1
(43) Pub. Date: Jan. 8, 2026

(54) PROTEIN PURIFICATION

(71) Applicant: NUCLERA LTD, Cambridge, Cambridgeshire (GB)

(72) Inventor: Michael Chun Hao Chen, Cambridge, Cambridgeshire (GB)

(21) Appl. No.: 18/881,807

(22) PCT Filed: Jul. 14, 2023
(86) PCT No.: PCT/GB2023/051865

§ 371 Date: Jan. 7, 2025

(30) Foreign Application Priority Data
Jul. 14, 2022 (GB) 2210384.0

(51) Int. Cl. C07K 1/22; C12P 21/02; G01N 33/68

(52) U.S. Cl. CPC C07K 1/22; C12P 21/02; G01N 33/6839; C07K 2319/21; C07K 2319/22; C07K 2319/60

(57) ABSTRACT

The invention provided herein relates to methods for protein synthesis, purification and characterisation.

### FIELD OF THE INVENTION

Provided herein are methods for purification of proteins or other biomolecules.

### BACKGROUND

Proteins are biological macromolecules that maintain the structural and functional integrity of the cell, and many diseases are associated with protein malfunction. Protein purification is a fundamental step for analysing individual proteins and protein complexes and identifying interactions with other proteins, DNA or RNA. A variety of protein purification strategies exist to address desired scale, throughput and downstream applications. However, protein production can be challenging for many reasons. One major challenge is finding a suitable expression system, for example sourced from mammalian, bacterial, fungal, or plant cells. This can take months of work.

Purification of proteins using functionalized magnetic beads (superparamagnetic particles, SPMP) is well known in the literature. One common method is to express a protein with a particularly tag sequence, for example a His tag (typically 6× histidine amino acids at either the N- or C-terminus) and isolate this protein from lysed cells using Ni-NTASPMP. Another common method is to express a protein with a Strep or Strep II tag (WSHPQFEK or AWAHPQPGG amino acid tags at N- or C-terminus) and isolate this protein from lysed cells using Streptavidin (or a related derivative) SPMP. Many types of tag sequences or binding moieties are known.

Cell-free protein synthesis, also known as in-vitro protein synthesis or CFPS, is the production of peptides or proteins using biological machinery in a cell-free system, that is, without the use of living cells. The in-vitro protein synthesis environment is not constrained within a cell wall or limited by conditions necessary to maintain cell viability, and enables the rapid production of any desired protein from a nucleic acid template, usually plasmid DNA or RNA from an in-vitro transcription. CFPS has been known for decades, and many commercial systems are available. Cell-free protein synthesis encompasses systems based on crude lysate (Cold Spring Harb Perspect Biol. 2016 December; 8 (12): a023853) and systems based on reconstituted, purified molecular reagents, such as the PURE system for protein production (Methods Mol Biol. 2014; 1118:275-284). CFPS requires significant concentrations of biomacromolecules, including DNA, RNA, proteins, polysaccharides, molecular crowding agents, and more (Febs Letters 2013, 2, 58, 261-268).

WO2022/038353 discloses method of expressing and analysing proteins on an electrophoresis device. The assays described therein can be used to determine expression levels, but not purity of the expressed proteins.

US20160230203 relates to methods of protein production, including bead based affinity purification and characterisation modules. The analysis modules described rely on electrophoresis or similar staining gels.

To date protein purification and analysis typically requires complex analysis techniques involving electrophoresis. The inventors herein have developed protein purification methods allowing multiple ways of characterising expressed proteins without the need for electrophoresis or other gel based separation methods.

### SUMMARY OF THE INVENTION

The invention relates to the synthesis, purification and characterisation of proteins. Following protein expression, a system of purification and characterisation of the resultant biomolecules is required.

Disclosed is a method for protein synthesis comprising expressing a protein in a cell-free system wherein the expressed protein contains a sub-component of a fluorescent protein, the method comprising measuring the yield of expressed protein using a fluorescence measurement, purifying the protein by affinity purification and measuring the total protein content of the purified protein.

Disclosed is a method for protein synthesis comprising expressing a protein in a cell-free system wherein the expressed protein contains a sub-component of a fluorescent protein, the method comprising measuring the yield of expressed protein using a fluorescence measurement, purifying the protein by affinity purification and measuring purified yield of the purified protein and the total protein content of the purified protein.

Disclosed is a method for protein synthesis comprising expressing a protein in a cell-free system wherein the expressed protein contains a sub-component of a fluorescent protein, the method comprising measuring the yield of expressed protein using a fluorescence measurement, purifying the protein by affinity purification, releasing the purified protein into solution and measuring the amount of expressed target protein and the total amount of protein in the solution.

The fluorescence measurement can be performed by assembly of a fluorescent protein from sub-components. The yield of expressed protein may be determined by fluorescence complementation. The expressed protein may contain GFP₁₁. The expressed protein may contain ccGFP₁₁.

The purification results in immobilisation of the protein. The immobilisation can use beads, for example magnetic or paramagnetic beads. After affinity binding, the proteins may be washed to remove non-specifically bound material. The protein may then be eluted into solution after purification.

The expression can be performed using cell lysates. The expression is performed using cell-free lysates or using assembled components for transcription and translation in a system of purified recombinant elements (PURE).

The term affinity binding refers to the immobilisation or capture of the protein via an interaction with a solid support. The affinity binding may be based on charge or hydrophobicity. The affinity binding may be based on having a particular amino acid sequence in the expressed protein. The affinity purification may use binding tags selected from:

Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(AWAHPQPGG) (AWRHPQFGG)
Strep-tag II
(WSHPQFEK)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

Multiple steps of purification may be performed. For example using multiple different binding tags, or using a binding tag and a hydrophobic or charge based interaction. The multiple purification staps can be performed sequentially in any order.

Once purified from the expression reagents, an assay to determine the total amount of purified protein may be performed. The assay for total protein may use a colourimetric based stain such as Coomassie. The assay is performed without requiring a gel based separation or staining.

The assays may be performed in small volumes, for example on a microtitre or strip plate. The assays may be performed in droplets on a microfluidic device, such as for example a digital microfluidic device. The digital microfluidic device may comprise an oil-filled or humidified gaseous environment, wherein the humidified gaseous environment is achieved by enclosing or sealing the digital microfluidic device and providing on-board reagent reservoirs.

There the assays are performed in droplets, droplets having the protein staining agent, for example Coomassie, can be added to the expressed and purified proteins. Suitable controls may include a set of droplets which have not been purified and a set of droplets having known protein concentrations. Droplets on a microfluidic device can be split or merged as desired.

The screening step can identify the optimal conditions for expression and purification of the desired protein.

Disclosed is a method comprising:
a. using a variety of different conditions to synthesise a protein of interest having a tag, thereby identifying the optimal conditions for the expression of soluble protein having the tag;
b. capturing the proteins via affinity to magnetic beads, thereby immobilising the proteins;
c. washing the beads;
d. eluting the protein from the beads; and
e. determining the level of the correctly expressed tag and the total amount of eluted protein.

Disclosed is a method comprising:
a. using a variety of different conditions to synthesise and purify a protein of interest having a tag, thereby identifying the optimal conditions for the expression and purification of soluble protein having the tag by measuring the total concentration of purified protein and the yield of expressed protein to determine the expressed yield and purity of the synthesised protein.

Also disclosed is a protein having both a sequence which contains a sub-component of a fluorescent protein and a sequence for affinity purification.

For the method or protein, the protein may contain a GFP₁₁ peptide amino sequence tag selected from:

KRDHMVLLEFVTAAGITGT
KRDHMVLHEFVTAAGITGT
KRDHMVLHESVNAAGIT
RDHMVLHEYVNAAGIT
GDAVQIQEHAVAKYFTV
GDTVQLQEHAVAKYFTV
GETIQLQEHAVAKYFTE

or a truncated version thereof. Truncations may involve a shortening of up to 5 amino acids from the N terminus, the C terminus or a combination thereof.

The method or protein, may contain a GFP₁₁ peptide amino sequence tag selected from:

KRDHMVLLEFVTAAGITGT
KRDHMVLHEFVTAAGITGT
KRDHMVLHESVNAAGIT
RDHMVLHEYVNAAGIT
GDAVQIQEHAVAKYFTV
GDTVQLQEHAVAKYFTV
GETIQLQEHAVAKYFTE

or a truncated version thereof,
in combination with binding tag selected from

Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(AWAHPQPGG) (AWRHPQFGG)
Strep-tag II
(WSHPQFEK)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

The protein may have both a GFP₁₁ region and a His tag. The protein may have both a GFP₁₁ region and a strep-tag or Strep-tag II.

Disclosed is a composition comprising reagents for cell-free protein synthesis, beads for purification of expressed proteins and reagents for measuring the total purified protein. The reagents for measuring total purified protein may comprise Coomassie, Bicinchoninic acid or NanoOrange®.

### BRIEF DESCRIPTION OF THE DRAWINGS

FIG. 1: A graph showing seven different protein samples measured in three ways, BCA, fluorescent complementation and gel based.

FIG. 2: Sections from 4 separate gels showing the purity of the proteins being measured in graph 1. The numbered columns show the lanes analysed. The adjacent columns show the expressed proteins prior to purification.

### DETAILED DESCRIPTION OF THE INVENTION

Disclosed is a method for protein synthesis comprising expressing a protein in a cell-free system wherein the expressed protein contains a sub-component of a fluorescent protein, the method comprising measuring the yield of expressed protein using a fluorescence measurement, purifying the protein by affinity purification and measuring the total protein content of the purified protein.

The method avoids the need for complex purification steps or gel based separations. A fluorescence based assay can be used to measure the amount of expressed protein (expressed yield). In conjunction with an assay measuring total protein gives the purity (i.e. how much protein is present. Thus both yield and purity can be determined after expression without needing to run gels.

The fluorescence measurement can act as a real time measure of protein expression. As the tag sequence is produced, the presence of the remaining fluorescent protein as a detector species then allows real-time measurement of the tag sequence. Alternatively the fluorescence can be measured by adding the detector species after expression. The fluorescence can be retained and monitored during purification. Alternatively further detector protein can be added after purification to determine the purified yield. Both yield of synthesis and efficiency of purification can thus be determined. For example GFP₁₁ can be attached to the expressed protein and GFP₁₋₁₀ used as the detector species.

This screening workflow enables users to rapidly screen different expression systems in the form of cell-free lysates. Having identified an optimal expression system, a scientist typically wants to obtain small quantities of protein to perform initial tests, for instance to verify protein molecular weight, solubility, and function (whether activity or binding affinity). To perform these tests a pure protein is typically required, hence there is a need for a method to separate a protein of interest from a complex mixture containing other proteins, nucleic acids, and other cellular components on a digital microfluidic device.

The affinity purification is based on a property of the expressed protein. The expressed protein can be purified via immobilisation. The immobilisation can be based on for example charge, hydrophobic interactions or based on specific sequence interactions such as binding of Protein A.

The protein can be expressed with a binding tag. The tag binding moiety can be incorporated during biopolymer synthesis or can be attached via conjugation after biopolymer synthesis. The binding tags may be attached via coupling to a functionalizable moiety on the polymer. The conjugation can be for example via chemical attachment such as for example via click chemistry using an azide/alkyne. The conjugation can be performed for example using a specific amino acid, which could be a natural or unnatural amino acid. The expressed translated protein may have an amino acid allowing protein modification post translation, for example a cysteine or lysine which can be chemically reacted with the tag sequence.

Immobilisation can be performed using magnetic beads. The beads may be functionalised with NTA (to bind Ni²⁺, Tb³⁺) or modified to immobilize streptavidin.

The binding moiety can be a region of amino acid/peptide sequence. The affinity binding site can be a region of amino acid/peptide sequences specific to a particular antibody. For example the binding moiety can be selected from the list of exemplary peptide affinity binding sites below:

Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(AWAHPQPGG) (AWRHPQFGG)
Strep-tag II
(WSHPQFEK)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

A single “tag” can be used for detection and for purification to prevent the encoded payload in the expression cassette becoming too large or to prevent multiple tags from interfering with the function of the biomolecule. In particular examples the tag can also be used to detect the presence of the biopolymer. Thus the tag can be used for the dual purpose of both detection and purification. This is advantageous in preventing the biopolymer becoming too large. In the case of proteins, addition of too much “exogenous” protein fused to either the N or C terminus of the protein can sometimes change the activity or function of the protein.

The binding moiety tag can be a sub-component of a fluorescent protein. Thus the fully assembled protein becomes fluorescent. For example if the immobilised material contains GFP₁₋₁₀ and the tag contains a GFP₁₁ peptide, complementation forms immobilised fluorescent GFP, allowing simultaneous monitoring and purification. The immobilised material can be washed and then eluted by disrupting the complemented split GFP, e.g. through the use of salt or temperature.

The binding moiety may include a small molecule affinity tag such as biotin. The binding moiety may include a particular sequence of nucleic acids.

The release of the immobilised biomolecules may be via cleavage of the tag or via disruption of binding of the tag to the support. The disruption may be via a change of buffer. The buffer may contain agent which disrupts binding, for example imidazole for His/Ni NTA or desthiobiotin for Strep tag/Streptavidin. The disruption may be via a change in temperature.

The process can be performed in droplets, which can be manipulated by electrokinesis in order to effect and improve protein purification. The droplet can be moved using any means of electrokinesis. The droplet can be moved using electrowetting on dielectric (EWoD). The electrical signal on the EWoD or optical EWoD device can be delivered through segmented electrodes, active-matrix thin-film transistors, or digital micromirrors.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

Any particular nucleic acid template can be expressed using the system described herein. Three types of nucleic acid templates used in cell-free protein synthesis (CFPS) include plasmids, linear expression templates (LETs), and mRNA. Plasmids are circular templates, which can be produced either in cells or synthetically. LETs can be made via PCR. mRNA can be produced through in-vitro transcription systems. The methods can use a single nucleic acid template per droplet. The methods can use multiple nucleic acid templates per drop. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The use of a population of droplets having different components allows the rapid screening of a variety of variable factors to identify optimal conditions for expression of a desired proteins. The protein can be contained with a sequence having other amino acid domains, for example solubility factors or binding tags.

Protein sequences disclosed herein may be attached to further elements to improve solubility. The variant may be attached to one or more solubility enhancing sequences. The solubility enhancing sequence may be a peptide sequence or a naturally occurring sequence. The solubility enhancing sequence may be selected from for example maltose binding protein (MBP), Small Ubiquitin-like Modifier (SUMO), Glutathione S-transferase (GST) or thioredoxin (TRX). The tags may be attached to either the C or N terminus. Any example of a solubility enhancer may be used. A list of possible proteins is shown below. Any sequence selected from the list below may be chosen:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

Any fluorescent protein may be used. The fluorescent protein may be sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide CFAST in the presence of a hydroxybenzylidene rhodanine analog. The peptide tag may be ccGFP₁₁ and the further polypeptide ccGFP 1-10.

The fluorescent protein may be GFP. The fluorescent protein may be sfGFP. The fluorescent protein may be ccGFP.

The protein may be assembled and thereby become fluorescent as a result of the expressed protein binding with the binding partner. The affinity interaction results in the two sub-components of the fluorescent protein being near enough to each other to bind and induce fluorescence.

The complementary GFP₁₁ peptide amino acid sequence tag could be the following:

1.
KRDHMVLLEFVTAAGITGT
2.
KRDHMVLHEFVTAAGITGT
3.
KRDHMVLHESVNAAGIT
4.
RDHMVLHEYVNAAGIT
5.
GDAVQIQEHAVAKYFTV
6.
GDTVQLQEHAVAKYFTV
7.
GETIQLQEHAVAKYFTE

or a truncated version thereof. Truncations may involve a shortening of up to 5 amino acids from the N terminus, the C terminus or a combination thereof.

Properties of the expressed protein may be characterised on the device. An initial screen may be based on the level of soluble expression by measuring fluorescence formed on complementation of a detector with the expressed sequence. The protein may remain fluorescent during immobilisation, at which point the level of affinity purification can be determined.

Further assays can be used to determine the total amount of protein present in a droplet. For example a whole protein determination assay such as the Bradford assay, which is a colorimetric protein assay based on an absorbance shift of the dye Coomassie. The assay can be performed in the same droplet or in parallel droplets. Thus droplets can be expressed in parallel, with one or more being used to measure the binding of soluble protein and different droplets used to measure total protein bound to the bead. The ratio of soluble protein having the correctly expressed tag to total protein isolated gives an indication of the purity of the isolated protein.

A large number of assays are available for measuring total protein in a particular sample. Suitable assays are described below:
https://www.thermofisher.com/uk/en/home/life-science/protein-biology/protein-assays-analysis/protein-assays.html

Assays such the total protein determination assay can be performed on the device, but preferably are performed after removal of the bulk of the expression reagents, which would otherwise dominate measures of total protein. The fluorescence assay for presence of expressed tags can be performed during the process of expression, or can be performed as an end-point by addition of the relevant detector.

Described are assays that measure both purity and levels of expression yield. Data useful for purification would be both recovered yield of soluble protein (mg/mL) for each purification step and the purity of the recovered protein. The total protein quantification can be performed using an assay such as a Bradford, Lowry or BCA assay, for example as listed below:
https://www.thermofisher.com/uk/en/home/life-science/protein-biology/protein-assays-analysis/protein-assays/protein-assay-selction-guide.html

% Purity=soluble mg/ml*100/total mg/ml

The assays can be performed in parallel in discreet reactions, or serially within the same reactions volume.

Assays for measuring total protein levels may involve the addition of colourimetric or fluorescent staining indicators. Suitable indicators include Coomassie (Bradford assay), Bicinchoninic acid (BCA) or fluorescence based assays for example NanoOrange®. In each case the indicator is added to the protein in order to measure total protein levels. The NanoOrange® protein quantification assay employs a merocyanine dye that increases fluorescence in the presence of proteins.

In addition to screening for the best conditions for expression, the method can also be used to screen the best conditions for purification of particular proteins. A protein of interest can be made with a variety of different amino acid appendages acting as affinity agents. The expressed amino acids can be exposed to a variety of different beads and the amount of bound material determined. A variety of washing steps can be performed. Thus as well as screening for efficient synthesis, efficient purification conditions can also be identified. Once identified, the optimal conditions and beads can be used for scale-up and purification.

As a hypothetical example, a variety of nucleic acid templates can be screened for expression. The best conditions may give say 20 micrograms of protein having the correct tag. A variety of purification conditions can be screened. The total amount of recovered material having the expressed tags may be say 14 micrograms, hence giving a protein recovery of 14/20. However the recovery may give 25 micrograms of total recovered protein, i.e. 13 micrograms of protein which does not contain the correct tag. Thus the purity would be 14/25. More efficient washing could be performed to improve the purity, ideally without lowering the 14 micrograms of correct material.

The screening and analysis can be performed in liquid reagent volumes, for example in microtitre plates or strip-tubes. Reagent volumes can be split such that portions are tested and portions retained for further use.

Such screening, characterising and purification can all be performed on a single device, which may be a digital microfluidic device. The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplets to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

The droplets can be aqueous droplets. The droplets can contain an oil immiscible organic solvent such as for example DMSO. The droplets can be a mixture of water and solvent, providing the droplets do not dissolve into the bulk oil.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWOD device can be delivered through segmented electrodes, active-matrix thin-film transistors or digital micromirrors. Optically-activated s-Si EWOD devices are well known in the art for actuating droplets (J. Adhes. Sci. Technol., 2012, 26, 1747-1771).

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated.

The droplet can be formed before entering the microfluidic device and flowed into the device. Alternatively the droplets can be merged on the device. Included is a method comprising merging a first droplet containing a nucleic acid template such as a plasmid with a second droplet containing a cell-free system having the components for protein expression to form the droplet.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting the lysate results in a decrease in expression level of the protein of interest. Thus performing CFPS on DMF where the surfactants are added to the solutions being moved will necessarily result in a dilution of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell-free system with a GFP₁₁ (or similar) peptide tag, it's downstream complementation with a GFP 1-10 detector polypeptide is hindered in the presence of surfactant.

Rather than adding surfactants to the aqueous sample, it is instead possible to add surfactant, such as Span85 (sorbitan trioleate), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the split GFP system, so removing surfactants from the reagent mix and instead adding them to the oil can be beneficial.

The oil in the device can be any water immiscible liquid. The oil can be mineral oil, silicone oil such as dodecamethylpentasiloxane (DMPS), an alkyl-based solvent such as decane or dodecane, or a fluorinated oil. The oil can be oxygenated prior to or during the expression process. Alternatively, the device can be an air-filled device where droplets containing cell-free protein synthesis reagents are rapidly moved into position and fixed into an array under a humidified gas to prevent evaporation. Humidification can be achieved by enclosing or sealing the digital microfluidic device and providing on-board reagent reservoirs. Additionally, humidification can be achieved by connecting an aqueous reservoir to an enclosed or sealed digital microfluidic device. The aqueous reservoir can have a defined temperature or solute concentration in order to provide specific relative humidities (e.g., a saturated potassium sulfate solution at 30° C.).

Also disclosed is a protein having both a sequence which contains a sub-component of a fluorescent protein and a sequence for affinity purification. The protein may contain GFP₁₁. The protein may contain a GFP₁₁ peptide amino sequence tag selected from:

KRDHMVLLEFVTAAGITGT
KRDHMVLHEFVTAAGITGT
KRDHMVLHESVNAAGIT
RDHMVLHEYVNAAGIT
GDAVQIQEHAVAKYFTV
GDTVQLQEHAVAKYFTV
GETIQLQEHAVAKYFTE

or a truncated version thereof. Truncations may involve a shortening of up to 5 amino acids from the N terminus, the C terminus or a combination thereof.

The protein, may contain a GFP 11 peptide amino sequence tag selected from:

KRDHMVLLEFVTAAGITGT
KRDHMVLHEFVTAAGITGT
KRDHMVLHESVNAAGIT
RDHMVLHEYVNAAGIT
GDAVQIQEHAVAKYFTV
GDTVQLQEHAVAKYFTV
GETIQLQEHAVAKYFTE
or
a truncated version thereof,

in combination with binding tag selected from

Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(AWAHPQPGG) (AWRHPQFGG)
Strep-tag II
(WSHPQFEK)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

The protein may have both a GFP₁₁ region and a His tag. The protein may have both a GFP₁₁ region and a strep-tag or Strep-tag II.

Additionally the protein can have a region for solubility enhancement, for example selected from maltose binding protein (MBP), Small Ubiquitin-like Modifier (SUMO), Glutathione S-transferase (GST) or thioredoxin (TRX) or those listed above.

Disclosed is a kit comprising reagents for cell-free protein synthesis, beads for purification of expressed proteins and reagents for measuring the total purified protein. The reagents for measuring total purified protein may comprise Coomassie, Bicinchoninic acid or NanoOrange®. The composition may be in the form of discreet droplets on a microfluidic device such as an electrowetting device. The device may be a digital microfluidic device as described herein.

An exemplary method may include the following steps:
1. Express the proteins bearing a purification tag (e.g. His, Strep) and a fluorescent sub-component using cell-free lysates. Monitor fluorescence using a suitable detector protein having the remaining fluorescent protein to determine the level of soluble protein produced.
2. Contact the reaction containing protein expressed in a cell-free lysate with superparamagnetic beads bearing a purification moiety (e.g. Ni-NTA for His tag, Streptavidin or an analogue thereof for Strep tag) and incubate for a period of time to enable the tag and purification moiety to interact.
3. Apply a magnetic field (e.g. by bringing a magnet into proximity with the digital microfluidic array or turning on an electromagnet) to pellet the superparamagnetic particles.
4. Remove the supernatant droplet from the pelleted superparamagnetic particles and then wash the superparamagnetic particles. The superparamagnetic particles may be resuspended in the wash reagents and then re-pelleted prior to removal of the liquid; this wash process may be repeated multiple times, which may increase the protein purity obtained.
5. Elute the tagged protein from the purification moiety on the superparamagnetic particles by contacting them with an elution solution (e.g. imidazole for His tag/Ni NTA or desthiobiotin for Strep tag/Streptavidin).
6. Measure the total protein eluted from the beads using for example a Bradford assay or BCA assay.

The amount of eluted tagged protein can also be measured, for example by repeating the fluorescence complementation measurement.

### EXAMPLE

Aim: To compare different techniques to quantify the elutions from protein expression and scale-up in order to have information related to the efficiency of purification and protein yields. Three techniques were compared: BCA assay for protein concentration, fluorescence based complementation and coomassie based gel staining.

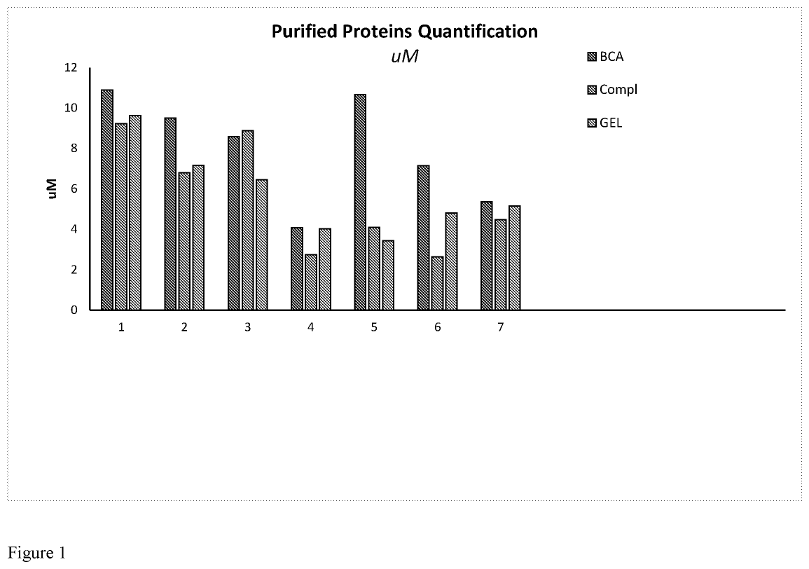

Seven proteins were expressed (labelled 1-7 in FIG. 1). Each protein contains both a ccGFP₁₁ detector tag sequence, and a strep purification tag:
1: MBP-DET-STREP
2/3: P17-MBP-DET-STREP
4/5: MOCR-MBP-DET-STREP
6/7: ZZ-STREP-MBP-DET

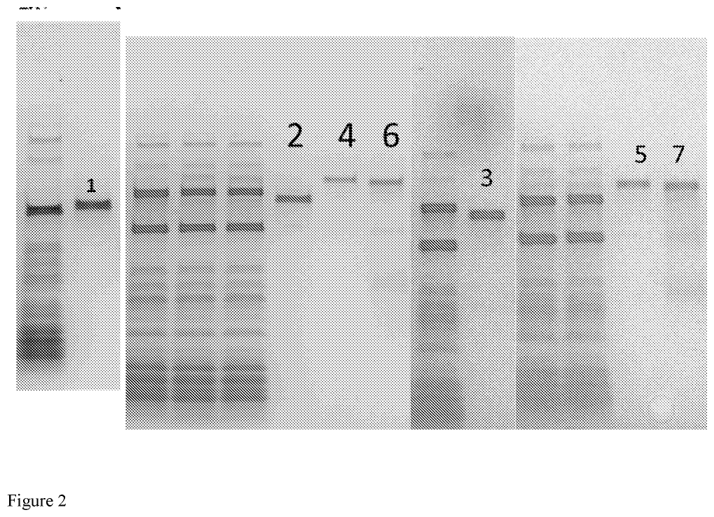

Each protein was expressed using a reconstituted using a cell-free protein expression system and purified using commercial beads containing a strep-tag binding protein. The crude and purified materials were run on a electrophoresis gel and stained with Coomassie in order to determine concentration and purity. Gel images are shown in FIG. 2. The band intensity for the purified material was measured against control bands of known protein concentration (not shown). The purified materials were also measured using fluorescence complementation to a protein detector (ccGFP₁₋₁₀) and measured against a standard ccGFP₁₋₁₁ control at known concentrations and also using a commercial BCA assay protocol measuring absorbance at 480 nm (Pierce Rapid Gold BCA Protein Assay for Determining Protein Concentration).

The three measurements for each sample are shown in FIG. 1. The BCA and fluorescence concentrations show the level of purity. Where samples are less pure, a higher level of BCA ‘total’ protein than gfp compliment is seen due to the presence of proteins which do not carry the ccGFP₁₁ detector tag. In samples where the bands in the gel are clean, showing high protein purity, the concentration levels of BCA and ‘GFP compl’ measurements correlate. Thus the ratio of expressed material carrying the GFP tag to the amount of total protein in the sample can be determined.

1. A method for protein synthesis comprising expressing a protein in a cell-free system wherein the expressed protein contains a sub-component of a fluorescent protein, the method comprising measuring the yield of expressed protein using a fluorescence measurement, purifying the protein by affinity purification, releasing the purified protein into solution and measuring the amount of expressed target protein and the total amount of protein in the solution.

2. The method according to claim 1, wherein the purified yield of protein is determined by fluorescence complementation.

3. The method according to claim 2, wherein the expressed protein contains a tag being a component of a fluorescent protein.

4. The method according to claim 2, wherein the expressed protein contains ccGFP₁₁.

5. The method according to any one of claims 1 to 4, wherein the affinity purification uses beads.

6. The method according to claim 5, wherein the affinity purification uses magnetic or paramagnetic beads.

7. The method according to any one of claims 1 to 6, wherein the expression is performed using cell-free lysates.

8. The method of claim 7, wherein the expression is performed using assembled components for transcription and translation in a system of purified recombinant elements (PURE).

9. The method according to any one of claims 1 to 8, wherein the affinity purification uses binding tags selected from:
Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(AWAHPQPGG) (AWRHPQFGG)
Strep-tag II
(WSHPQFEK)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

10. The method according to any one of claims 1 to 9, wherein the immobilised protein are washed to further purify.

11. The method according to any one of claims 1 to 10, wherein the assay to determine the total protein content uses Coomassie, Bicinchoninic acid or NanoOrange®.

12. The method according to any one of claims 1 to 11, wherein the method is performed on a digital microfluidic device.

13. The method according to claim 12 wherein the digital microfluidic device comprises an oil-filled or humidified gaseous environment, wherein the humidified gaseous environment is achieved by enclosing or sealing the digital microfluidic device and providing on-board reagent reservoirs.

14. The method according to any one of claims 1 to 13 wherein a screening step identifies the optimal conditions for expression of the desired protein.

15. The method according to claim 1 comprising
a. using a variety of different conditions to synthesise a protein of interest having a tag, thereby identifying the optimal conditions for the expression of soluble protein having the tag;
b. capturing the proteins via affinity to magnetic beads, thereby immobilising the proteins;
C. washing the beads;
d. eluting the protein from the beads; and
e. determining both the level of the correctly expressed tagged protein and the total amount of eluted protein.

16. The method according to claim 1 comprising
a. using a variety of different conditions to synthesise and purify a protein of interest having a tag, thereby identifying the optimal conditions for the expression and purification of soluble protein having the tag by measuring the total concentration of purified protein and the yield of expressed protein to determine the expressed protein yield and purity of the synthesised protein.

17. A protein having both a sequence which contains a sub-component of a fluorescent protein and a sequence for affinity purification.

18. The method according to any one of claims 1 to 16 or a protein according to claim 17 wherein the protein contains a ccGFP₁₁ peptide amino sequence tag selected from:
KRDHMVLLEFVTAAGITGT
KRDHMVLHEFVTAAGITGT
KRDHMVLHESVNAAGIT
RDHMVLHEYVNAAGIT
GDAVQIQEHAVAKYFTV
GDTVQLQEHAVAKYFTV
GETIQLQEHAVAKYFTE
or a truncated version thereof, and a binding tag selected from
Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
lanthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(AWAHPQPGG) (AWRHPQFGG)
Strep-tag II
(WSHPQFEK)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

19. The protein according to claim 18 having a ccGFP₁₁ region and a His, strep-tag or strep-II tag.

20. The protein according to claim 18 or claim 19 further having a region for solubility enhancement.

21. The protein according to claim 20 wherein the solubility region is selected from maltose binding protein (MBP), Small Ubiquitin-like Modifier (SUMO), Glutathione S-transferase (GST), thioredoxin (TRX), T7 phage tail (P17), metal-binding protein (CUSF), 53-amino-acid-long N-terminal extension sequence (NEXT), Fasciola hepatica 8 kDa antigen (FH8), Solubility Enhancing Ubiquitous Tag (SNUT) or IgG repeat domain ZZ of Protein A (ZZ).

22. A kit comprising reagents for cell-free protein synthesis, beads for purification of expressed proteins and reagents for measuring the total purified protein comprising Coomassie, Bicinchoninic acid or NanoOrange®.
